(19) United States
(12) Patent Application Publication
(10) Pub. No.: US 2023/0383359 A1
(43) Pub. Date: Nov. 30, 2023

(54) METHODS FOR TREATMENT OF LUNG CANCERS

(71) Applicant: Mirati Therapeutics, Inc., San Diengo, CA (US)

(72) Inventors: James Gail Christensen, San Diego, CA (US); Andrew Chi, La Jolla, CA (US); Thian Kheoh, San Diego, CA (US)

(21) Appl. No.: 18/032,492

(22) PCT Filed: Oct. 21, 2021
(86) PCT No.: PCT/US2021/056062

§ 371 Date: Apr. 18, 2023

Related U.S. Application Data
(60) Provisional application No. 63/105,091, filed on Oct. 23, 2020.

(51) Int. Cl. C12Q 1/6886; A61K 31/519; A61K 45/06; A61P 35/00

(52) U.S. Cl. CPC C12Q 1/6886; A61K 31/519; A61K 45/06; A61P 35/00; C12Q 2600/106; C12Q 2600/156

(57) ABSTRACT

The present invention relates to methods for treatment of lung cancers, in particular non-small cell lung cancer (NSCLC), wherein lung cancers have both a KRAS G12C mutation and an STK11 mutation, with adagrasib (MRTX849).

### FIELD OF THE INVENTION

The present invention relates to methods for treatment of lung cancers, in particular, non-small cell lung cancer (NSCLC), wherein lung cancers have both a KRAS G12C mutation and an STK11 mutation.

### BACKGROUND OF THE INVENTION

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors regulating a wide variety of processes, including cellular proliferation (e.g., see Alamgeer—2013).

The role of activated KRas in malignancy was observed over thirty years ago (e.g., see Santos—1984). Aberrant expression of KRas accounts for up to 20% of all cancers and oncogenic KRAS mutations that encode variant KRas proteins that stabilize GTP binding and lead to constitutive activation of KRas and downstream signaling have been reported in 25-30% of lung adenocarcinomas (e.g., see Samatar—2014). Of the KRAS mutations reported in lung adenocarcinomas, single nucleotide nonsynonymous missense mutations that result in single amino acid replacements in codons 12 and 13 of the KRas primary amino acid sequence comprise approximately 40%, with a G12C amino acid replacement being the most common activating mutation (e.g., see Dogan—2012).

The well-known role of KRas in malignancy and the discovery of these frequent mutations in KRAS in various tumor types made KRas a highly attractable target of the pharmaceutical industry for cancer therapy. Notwithstanding thirty years of large scale discovery efforts to develop inhibitors of KRas for treating cancer, no KRas inhibitor has demonstrated sufficient safety and/or efficacy to obtain regulatory approval (e.g., see McCormick—2015).

Recently, irreversible, covalent inhibitors that target KRas G12C have been described (e.g., see Ostrem—2013). For instance, commonly-owned and assigned U.S. Provisional Application Ser. No. 62/586,775 discloses potent, orally bioavailable compounds that irreversibly bind to KRas G12C for treating KRAS G12C mutant cancers.

A covalent, irreversible inhibitor of KRas G12C is 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1 -(2-fluoroprop-2-enoyl)piperazin-2-yl]acetonitrile, also known as MRTX849 and adagrasib. An amorphous form of this compound was described in International Patent Application PCT/US2018/061060 filed Nov. 14, 2018, published as WO2019/099524A1 on May 23, 2019 at Example 478, and in Fell—2020. Crystalline forms of this compound were described in U.S. Provisional Application Ser. Nos. 63/077,553, filed Sep. 11, 2020 and 63/093,673, filed Oct. 19, 2020. The contents of all of the above-mentioned patent applications are hereby incorporated by reference in their entirety.

The STK11 gene also known as liver kinase B1 or LKB1, encodes the serine/threonine-protein kinase STK11 protein. STK11 is a tumor suppressor gene that is somatically mutated or deleted in many common types of cancer, including but not limited to lung adenocarcinomas (LUAD) (approximately 15%), non-melanoma skin cancer (approximately 5%), cholangiocarcinomas (approximately 3%), ovarian carcinomas (approximately 3%) and pancreatic adenocarcinomas (approximately 2%) (Sanchez-Cespedes—2002, Ji—2007, Gurumurthy—2010, Gill—2011, Cerami—2012, Gao—2013, Zehir—2017, Robinson—2017, Sanchez-Vega—2018). In addition, germline mutations of STK11 cause the autosomal dominant Peutz-Jeghers syndrome (Hemminki—1998, Jenne—1998), which is characterized by mucocutaneous melanin pigmentation, hamartomatous polyps in the gastrointestinal tract and significantly increased risk of cancer development in various tissues, the most common of which is gastrointestinal, as well as in breast, lung and gynecological tissues (Alessi—2006, Sanchez-Cespedes—2007).

The STK11 gene, located on human chromosome 19p13, includes nine coding exons and one noncoding exon and codes for the 433 amino acid serine/threonine-protein kinase STK11 protein (also known as LKB1 protein) which is widely expressed in all tissues (Hemminki—1998, Alessi—2006, Sanchez-Cespedes—2007). STK11 mutations found in cancer include point mutations or small indels and frequently co-occur with other STK11 genomic alterations such as copy number alteration or gene deletions. Point mutations of STK11 are frequently nonsense or frame shift mutations predicted to be deleterious and oncogenic (Chakravarty—2017), and these mutations along with STK11 gene deletion result in loss of serine/threonine-protein kinase STK11 (LKB1) protein expression or loss of wild-type serine/threonine-protein kinase STK11 (LKB1) protein activity.

The serine/threonine-protein kinase STK11 (LKB1) protein is a serine-threonine kinase that has a critical role in cellular energy metabolism as it is the key upstream activator of AMP-activated protein kinase (AMPK), a central regulator of cellular energy homeostasis that balances nutrient supply with energy demand. Under low ATP conditions, such as during nutrient deprivation or hypoxia, serine/threonine-protein kinase STK11 (LKB1) phosphorylates and activates AMPK (Zeqiraj—2009), which in turn phosphorylates and inactivates enzymes involved in the synthesis of macromolecules while promoting catabolism. Among the critical targets AMPK represses is mTOR complex 1 (mTORC1), which occupies a central role in controlling cell growth (Laplante—2009). In addition to its role as a master regulator of cell metabolism, serine/threonine-protein kinase STK11 (LKB1) is implicated in diverse cell functions such as energy stress responses, cell growth, cell polarity, epigenetic reprogramming, angiogenesis, extracellular matrix remodeling, and genomic instability (Wodarz—2007, Mihaylova—2011, Liu—2013, Li—2015, Kottakis—2016, Zhang—2017, Skoulidis—2019).

In cancer cells, loss-of-function mutations in STK11 result in aberrant activation of mTOR and reprogramming of metabolism and epigenetics to promote malignant phenotypes such as cell growth, proliferation, increased invasion and metastasis (Ji—2007, Shackelford—2009, Gurumurthy—2010, Kottakis—2016, Zhang—2017). In lung cancer, deletion of the STK11 gene is insufficient for lung cancer initiation in mouse models, however loss of the STK11 gene dramatically accelerates carcinogenesis and induces early metastatic dissemination in a KRAS mutant background (Ji—2007).

Loss-of-function STK11 mutations also significantly alter the tumor immune microenvironment. In KRAS mutant lung cancer models, STK11 gene loss increases expression of cytokines that in turn trigger a marked influx of tumor-associated neutrophils with T cell suppressive properties (Koyama—2016), and also induces epigenetic repression of STING to promote insensitivity to cytosolic double stranded DNA accumulation (Kitajima—2019). In human non-small cell lung cancer (NSCLC), STK11 mutations are associated with a cold, non-T cell-inflamed microenvironment, characterized by paucity of infiltrating CD3+, CD4+ and CD8+ T-cells and low tumor cell expression of PD-L1, despite intermediate to high tumor mutational burden (TMB) (Skoulidis—2015, Scheel—2016, Kadara—2017, Skoulidis—2018, Cristescu—2018). In an investigation of genomic driver mutations associated with absence of PD-L1 expression in LUAD, only STK11 mutation was significantly associated in PD-L1 negative tumors (Skoulidis—2018).

Recent large real-world datasets indicate that STK11 mutations are prognostic for poor progression-free survival (PFS) and overall survival (OS) in NSCLC. In a real-world cohort of NSCLC patients treated with first-line chemotherapy or anti-PD-1/PD-L1 therapy, STK11 mutations were associated with worse real-world progression-free survival (rwPFS) and OS compared to patients with wild-type STK11 and KEAP1 (a gene that is frequently co-mutated with STK11 in NSCLC patients) NSCLC (Papillon-Cavanagh—2020). There was no difference in outcomes between STK11 mutant patients who were treated with chemotherapy or anti-PD-1/PD-L1 therapy, suggesting STK11 mutation is prognostic and not predictive of treatment response. Another real-world dataset analysis demonstrated worse rwPFS and OS in patients with STK11 mutant vs. wild-type NSCLC who were treated with either first-line anti-PD-1/PD-L1 therapy or first-line chemotherapy (Shire—2020). Similarly, patients with KRAS and STK11 co-mutations treated at first-line with either anti-PD-1/PD-L1 therapy or chemotherapy had worse PFS and OS compared to patients with wild-type KRAS and STK11. These data indicate STK11 mutation is associated with poor prognosis however its predictive value for response to treatment is unclear.

However, recent clinical evidence exists indicating STK11 mutation is associated with PD-1/PD-L1 inhibitor resistance, in keeping with the prominent role of STK11 in shaping the tumor immune microenvironment. In a study of PD-L1 positive (defined as PD-L1 tumor proportion score >1%) nonsquamous NSCLC patients treated with PD-1/PD-L1 inhibitors, STK11 mutations were associated with significantly lower objective response rate (ORR) and dramatically shorter PFS and OS compared with wild-type STK11 (Skoulidis—2018). STK11 mutation has also been associated with primary resistance to combination chemo-immunotherapy with pemetrexed-carboplatin (or cisplatin)-pembrolizumab (Skoulidis—2019) and to dual immune checkpoint inhibition (anti-PD-1 and anti-CTLA-4) in the first line treatment setting of NSCLC patients (Hellman—2018).

Notably, more recent data indicate that the predictive negative effect of STK11 mutation on anti-PD-1/PD-L1 response may perhaps be restricted to the KRAS mutant subset of NSCLC. STK11 mutation occurs more frequently in KRAS or KRAS G12C mutant LUAD, occurring in ˜25-30% of KRAS mutant LUAD compared to ˜15% of all LUAD, and co-occurrence of KRAS and STK11 mutations appear to represent a distinct clinical and biological subset of LUAD (Cerami—2012, Papillon-Cavanagh—2020, Zehir—2017, Shire—2020). STK11 mutant NSCLC is clinically heterogeneous demonstrated by the observations that although STK11 mutations are associated with inferior outcomes in NSCLC, subsets of patients with STK11 mutations have experienced durable clinical benefit from pembrolizumab monotherapy and combination chemo-immunotherapy in the first line setting (Cho—2020, Gadgeel—2020). Mechanistically, loss of the STK11 gene promoted PD-1/PD-L1 inhibitor resistance in KRAS mutant mouse LUAD models, indicating a causal role (Skoulidis—2018), and co-occurrence of STK11 mutation defined a distinct biological subset of KRAS mutant LUAD characterized by a comparative lack of immune system engagement, including low PD-L1 expression (Skoudilis—2015).

Initial clinical evidence suggesting that the negative effect of STK11 mutation on PD-1 pathway inhibitor response is restricted to the KRAS mutant subset of NSCLC patients was a report from a single institution retrospective dataset of NSCLC patients treated with PD-1 inhibitors at various lines of treatment. Among KRAS mutant patients, those with STK11 concurrent mutation (n=50) had shorter median PFS and median OS compared to STK11 wild-type patients (PFS: 1.8 vs. 4.6 months, HR: 0.46 [95% CI: 0.32-0.67], P<0.0001; OS: 4.8 vs. 13.6 months, HR: 0.51 [95% CI: 0.34-0.76], P=0.001). STK11 mutation status did not, however, impact outcome in KRAS wild-type patients (Ricciuti—2019).

A post-hoc analysis of a first-line NSCLC trial comparing combination chemo-immunotherapy with the PD-L1 inhibitor atezolizumab, with or without bevacizumab, to chemotherapy with bevacizumab demonstrated comparatively greater clinical benefit in atezolizumab-containing arms in the subset with KRAS mutations alone versus the subset with KRAS and STK11 and/or KEAP1 mutations. Patients with KRAS mutations alone had longer PFS and OS in the atezolizumab-containing arms versus the arm without atezolizumab (PFS of 15.2 and 7.4 months in the atezolizumab-containing arms vs. 6.0 months in the arm without atezolizumab; OS of 26.2 and 21 months in the atezolizumab-containing arms vs. 10.7 months in the arm without atezolizumab). In the subgroup with KRAS and STK11 and/or KEAP1 co-mutations, there was a numerical improvement in PFS and OS in the atezolizumab and bevacizumab containing arm versus the arm without atezolizumab, however the magnitude was significantly smaller (PFS of 6.0 vs. 3.4 months; OS of 11.1 vs. 8.7 months). Notably, the atezolizumab with chemotherapy arm without bevacizumab had numerically worse PFS and OS compared to chemotherapy with bevacizumab (PFS of 3.2 vs. 3.4 months; OS of 7.9 vs 8.7 months) in this subgroup with KRAS and STK11 and/or KEAP1 co-mutation (West—2020). In another analysis of two independent clinical cohorts of KRAS mutant LUAD patients, KRAS and STK11 co-mutation was associated with lower objective response rates (ORR) with anti-PD-1/PD-L1 in 2nd and later lines of treatment compared to KRAS and TP53 co-mutation or KRAS mutation alone (0% for patients with KRAS and STK11 co-mutation vs. 57% and 18% for patients with KRAS and TP53 co-mutation or KRAS mutation alone in one cohort; 7% for patients with KRAS and STK11 co-mutation vs. 36% and 29% for patients with KRAS and TP53 co-mutation or KRAS mutation alone in the 2nd cohort). Additionally, patients with KRAS and STKI I co-mutation had significantly shorter PFS and OS versus patients with KRAS mutation only (1.9 vs. 2.7 months, HR 1.87, P<0.001 for PFS; 6.4 vs. 16.0 months, HR 1.99, P=0.0015) (Skoulidis—2018).

These data indicate that STK11 mutations are associated with a poor prognosis in LUAD. In the subset of LUAD with KRAS mutations, co-occurring STK11 mutations may predict for inferior clinical outcomes to anti-PD-1/PD-L1 therapy, which is the current standard-of-care for 1st and later lines of treatment for advanced/metastatic NSCLC.

Therefore, NSCLC with concurrent KRAS and STK11 mutations, for which there is a significant trend for co-occurrence in NSCLC (Cerami—2012, Papillon-Cavanagh—2020, Zehir—2017, Shire—2020), and which represent approximately 6-7% of all NSCLC patients (Zehir—2017, Shire—2020), represents a significant unmet medical need.

### SUMMARY OF THE INVENTION

In one embodiment, the invention provides a method of treating lung cancer in a subject in need thereof, comprising administering to the subject a therapeutically effective amount of 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1-(2-fluoroprop-2-enoyl)piperazin-2-yl]acetonitrile, or a pharmaceutically acceptable salt thereof, wherein the lung cancer has been determined to have a loss-of-function mutation in STK11 (LKB1), or loss of expression of serine/threonine-protein kinase STK11 (LKB1) protein, and wherein the lung cancer is a KRAS G12C mutant cancer.

In one embodiment, the lung cancer is a non-small cell lung cancer (NSCLC).

In one embodiment, the subject is a human.

In one embodiment, the human is an adult patient.

In another embodiment, the human is a pediatric patient.

In one embodiment, the therapeutically effective amount of 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1-(2-fluoroprop-2-enoyl)piperazin-2-yl]acetonitrile, or a pharmaceutically acceptable salt thereof, is between about 200 and 1200 mg twice per day (BID).

In one embodiment, the therapeutically effective amount of 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1-(2-fluoroprop-2-enoyl)piperazin-2-yl]acetonitrile, or a pharmaceutically acceptable salt thereof, is about 600 mg twice per day (BID).

In some embodiments of any of the methods described herein, before treatment with the compositions or methods of the invention, the patient was treated with one or more of a chemotherapy, a targeted anticancer agent, an immunotherapy agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with an immune checkpoint inhibitor, and optionally, the prior treatment with the immune checkpoint inhibitor was unsuccessful; and/or the patient has been treated with a kinase inhibitor, and optionally, the prior treatment with the kinase inhibitor was unsuccessful; and/or the patient was treated with one or more other therapeutic agent(s).

In one embodiment, the invention further comprises administering to the subject a second anti-cancer therapy. The second anti-cancer therapy may be selected from the group consisting of a surgery, an immunotherapy, a radiotherapy, a gene therapy, and a chemotherapy.

In one embodiment, the second anti-cancer therapy is an immune checkpoint inhibitor.

In one embodiment, the second anti-cancer therapy is a PD-1/PD-L1 inhibitor.

In one embodiment, the PD-1/PD-L1 inhibitor is a PD-1 inhibitor.

In one embodiment, the PD-1 inhibitor is nivolumab, pembrolizumab, cemiplimab or tislelizumab, or a biosimilar thereof.

In another embodiment, the PD-1/PD-L1 inhibitor is a PD-L1 inhibitor.

In one embodiment, the PD-L1 inhibitor is atezolizumab, avelumab, or durvalumab, or a biosimilar thereof.

In one embodiment, the PD-1/PD-L1 inhibitor and the KRas G12C inhibitor are administered on the same day.

In another embodiment, the PD-1/PD-L1 inhibitor and the KRas G12C inhibitor are administered on different days.

### BRIEF DESCRIPTION OF THE FIGURES

FIG. 1 is a bar graph illustrating best overall response of NSCLC patients in Mirati Study 849-001 with KRAS G12C and STK11 mutations who initiated treatment with adagrasib 600 mg twice a day (BID) monotherapy in Phase 1/1b and in the Phase 2 cohort of patients with KRAS G12C identified in tumor tissue.

FIG. 2 is a bar graph illustrating best overall response by STK11 mutation status in NSCLC patients in Mirati Study 849-001 who initiated treatment with adagrasib 600 mg BID monotherapy in Phase 1/1b and in the Phase 2 cohort of patients with KRAS G12C identified in tumor tissue.

### DETAILED DESCRIPTION OF THE INVENTION

The present invention relates to methods for treatment of lung cancers, in particular non-small cell lung cancer (NSCLC), wherein lung cancers have both a KRAS G12C mutation and an STK11 mutation.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, a “loss-of-function mutation” refers to a DNA mutation (e.g., a substitution, deletion, insertion, truncation, splice site, translation start site, fusion, or frameshift mutation) that results in expression of a mutant protein that no longer exhibits wild-type activity (e.g., reduced or eliminated wild-type biological activity or enzymatic activity), results in expression of only a fragment of the protein that no longer exhibits wild-type activity, or results in no expression of the wild-type protein. Herein, a loss-of-function mutation refers to one of the following:

1) a nonsense mutation, defined as a genetic alteration that causes the premature termination of a protein. The altered protein may be partially or completely inactivated, resulting in a change or loss of protein function;

2) a frameshift mutation, defined as an insertion or deletion involving a number of base pairs that is not a multiple of three, which consequently disrupts the triplet reading frame of a DNA sequence. Such variants (or mutations) usually lead to the creation of a premature termination (stop) codon, and result in a truncated (shorter-than-normal) protein product;

3) a splice-site mutation, defined as a genetic alteration in the DNA sequence that occurs at the boundary of an exon and an intron (splice site). This change can disrupt RNA splicing resulting in the loss of exons or the inclusion of introns and an altered protein-coding sequence;

4) a translation start site mutation, defined as a mutation that disrupts the translation initiation sequence and abolishes the initiation of translation at the normal start site, and consequently, results in loss of mRNA translation or translation of an abnormal messenger RNA (mRNA). This mutation results in loss of expression of the protein or synthesis of a protein with abnormal amino acid sequence;

5) a recurrent somatic mutation, defined as having at least 5 instances recorded in the Catalogue of Somatic Mutations in Cancer (COSMIC) database (Tate—2019);

6) a DNA fusion, defined as a gene made by joining parts of two different genes. Fusion genes, and the fusion proteins that come from them, may be made when part of the DNA from one chromosome moves to another chromosome;

7) a mutation predicted to have a deleterious functional impact on the encoded protein by the OncoKB algorithm (Chakravarty—2017) or MutationAssessor (Reva—2011);

8) is not a variant of unknown significance, defined as a variation in a genetic sequence for which the association with disease risk is unclear. Also called unclassified variant, variant of uncertain significance, and VUS (Richards—2015);

9) is not a germline variant, defined as a gene change in a reproductive cell (egg or sperm) that becomes incorporated into the DNA of every cell in the body of the offspring, identified in the dbSNP (Sherry—2001).

For example, a loss-of-function mutation affecting the STK11 gene in a cancerous cell may result in the loss of expression of the serine/threonine-protein kinase STK11 (LKB1) protein, expression of only a fragment of the serine/threonine-protein kinase STK11 (LKB1) protein, or expression of a serine/threonine-protein kinase STK11 (LKB 1) protein that exhibits diminished or no enzymatic activity (e.g., no serine/threonine kinase enzymatic activity) in the cancerous cell. Non-limiting examples of loss-of-function mutations have been observed affecting the STK11 gene that can result in loss of, dysfunctional or defective serine/threonine-protein kinase STK11 (LKB1), e.g., as described in Launonen—2005,; Zaba—2013; Johnson—2012; and Gill—2011.

As used herein, “STK11 mutation” refers to a loss-of-function mutation in the STK11 gene (HGNC symbol STK11, Ensembl ID ENSG00000118046.16), which encodes the human serine/threonine-protein kinase STK11 protein (UniProtKB/Swiss-Prot Q15831). Non-limiting examples of STK11 mutations that are loss-of-function mutations as defined herein are listed in Appendix 1. The STK11 mutations listed in Appendix 1 were extracted from cBioPortal (Cerami—2012) on 22 Oct. 2020. Data were extracted from the Zehir—2017 dataset included in cBioPortal and filtered to select for mutations that were predicted to have deleterious function by OncoKB or had at least 5 occurrences in COSMIC, and excluded mutations and copy number alterations of unknown significance (i.e., VUS) and germline mutations. Appendix 1 at the end of the specification contains a non-exhaustive and non-limiting list of STK11 loss-of-function mutations.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, “KRAS G12C” refers to a form of a KRAS gene (HGNC symbol KRAS, Ensembl ID ENSG00000133703.13) that contains a variation in a single nucleotide that results in an amino acid substitution of a cysteine for a glycine at amino acid position 12 of the human KRas protein. The assignment of amino acid codon and residue positions for human KRas is based on the amino acid sequence identified by UniProtKB/Swiss-Prot P01116: Variant p.Gly12Cys.

As used herein, “KRas G12C” refers to a mutant form of a mammalian KRas protein that contains an amino acid substitution of a cysteine for a glycine at amino acid position 12. The assignment of amino acid codon and residue positions for human KRas is based on the amino acid sequence identified by UniProtKB/Swiss-Prot P01116: Variant p.Gly12Cys.

As used herein, “Programmed cell death protein 1 (PD-1)” is a 55 kDa type I transmembrane protein that is part of the Ig gene superfamily that delivers negative cellular signals upon interaction with its two ligands, PD-L1 or PD-L2, to suppress the immune response.

As used herein, a “PD-1/PD-L1 inhibitor” refers to an agent that is capable of negatively modulating or inhibiting all or a portion of the PD-1/PD-L1 axis signaling activity and include agents that block PD-1 or PD-L1. Examples include PD-1 and PD-L1 binding antagonists such as anti-PD-1 antibodies, antigen binding fragments thereof, immunoadhesins, aptamers, fusion proteins, and oligopeptides. In some embodiments, the PD-1 binding antagonist is an anti-PD-1 antibody. In some embodiments, the PD-L1 binding antagonist is an anti-PD-L1 antibody.

The term “PD-1 binding antagonist” as used herein refers to a PD-1 inhibitor, i.e., a molecule that decreases, blocks, inhibits, abrogates or interferes with signal transduction resulting from the interaction of PD-1 with one or more of its binding partners, such as PD-L1 and/or PD-L2. In some embodiments, the PD-1 inhibitor is a molecule that inhibits the binding of PD-1 to its binding partners. In a specific aspect, the PD-1 inhibitor inhibits the binding of PD-1 to PD-L1 and/or PD-L2. For example, PD-1 inhibitors include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 inhibitor reduces the negative co-stimulatory signal mediated by or through cell surface proteins expressed on T lymphocytes mediated signaling through PD-1 so as render a dysfunctional T-cell less non-dysfunctional. In some embodiments, the PD-1 inhibitor is an anti-PD-1 antibody. In one embodiment, the PD-1 antibody is pembrolizumab, or a biosimilar thereof. In one embodiment, the PD-1 antibody is cemiplimab, or a biosimilar thereof. In one embodiment, the PD-1 antibody is tislelizumab, or a biosimilar thereof.

The term “PD-L1 binding antagonist” as used herein refers to a PD-L1 inhibitor, i.e., a molecule that decreases, blocks, inhibits, abrogates or interferes with signal transduction resulting from the interaction of PD-L1 with either one or more of its binding partners, such as PD-1 and/or B7-1. In some embodiments, a PD-L1 inhibitor is a molecule that inhibits the binding of PD-L1 to its binding partners. In a specific aspect, the PD-L 1 inhibitor inhibits binding of PD-L1 to PD-1 and/or B7-1. In some embodiments, the PD-L1 inhibitors include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one embodiment, a PD-L1 inhibitor reduces the negative co-stimulatory signal mediated by or through cell surface proteins expressed on T lymphocytes mediated signaling through PD-L1 so as render a dysfunctional T-cell less non-dysfunctional. In some embodiments, a PD-L1 inhibitor is an anti-PD-L1 antibody. In a specific aspect, an anti-PD-L1 antibody is avelumab or a biosimilar thereof. In another specific aspect, an anti-PD-L1 antibody is atezolizumab or a biosimilar thereof. In another specific aspect, an anti-PD-L1 antibody is durvalumab or a biosimilar thereof. In another specific aspect, an anti-PD-L1 antibody is BMS-936559 (MDX-1105) or a biosimilar thereof.

A “biosimilar” means an antibody or antigen-binding fragment that has the same primary amino acid sequence as compared to a reference antibody (e.g., nivolumab or pembrolizumab) and optionally, may have detectable differences in post-translation modifications (e.g., glycosylation and/or phosphorylation) as compared to the reference antibody (e.g., a different glycoform).

As used herein, a “complete response” or “CR” refers to a subject having a KRas G12C-associated lung cancer with an STK11 genomic alteration that has been treated with adagrasib and in which the treated cancer at some stage of treatment is no longer detectable by palpation, by calibration or by standard-of-care methodologies for detecting such cancers that eventually relapse.

As used herein, a “durable complete response” refers to a subject having a KRas G12C-associated lung cancer with an STK11 genomic alteration that has been treated with adagrasib in which the treated cancer is no longer detectable by palpation, by calibration or by standard-of-care methodologies for detecting such cancers and the cancer fails to relapse due to an induced anti-cancer immunological memory in the subject, remaining undetectable after treatment and/or in patient-derived animal models (PDX) is recalcitrant to re-challenge using the same cancer cells of the initial cancer type. The duration of a durable complete response is typically measured in weeks, months or years.

As used herein, a “partial response” or “PR” refers to the definition used in the Response Evaluation Criteria In Solid Tumors (RECIST) 1.1 criteria (Eisenhauer—2009) which provides “greater than or equal to 30% decrease under baseline of the sum of diameters of all target measurable lesions. The short diameter is used in the sum for target nodes, while the longest diameter is used in the sum for all other target lesions. All target lesions must be assessed.” A PR requires confirmation of the 30% decrease under baseline of the sum of diameters of all target measurable lesions on a disease assessment methodology at least 4 weeks after first observation of PR. An “unconfirmed PR” refers to an initial observation of PR on a disease assessment methodology that has not yet been confirmed on a follow-up disease assessment at least 4 weeks after first observation of PR.

As used herein, the terms “MRTX849” and “adagrasib” refer to a compound with the name 2-[(2 S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1-(2-fluoroprop-2-enoyl)piperazin-2-yl]acetonitrile An amorphous form of this compound was described in International Patent Application PCT/US2018/061060 filed Nov. 14, 2018, published as WO2019/099524A1 on May 23, 2019 at Example 478, and in Fell—2020. Crystalline forms of this compound were described in U.S. Provisional Application Ser. Nos. 63/077,553, filed Sep. 11, 2020 and 63/093,673, filed Oct. 19, 2020. All these forms are encompassed by the methods of the present invention.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a lung cancer having both a KRAS G12C mutation and a STK11 mutation (e.g., as determined using a regulatory agency-approved, e.g., FDA-approved, assay or kit). In some embodiments, the subject is suspected of having a KRAS G12C mutation and an STK11 mutation. In some embodiments, the subject has a clinical record indicating that the subject has a lung cancer having a KRAS G12C mutation and a STK11 mutation (and optionally the clinical record indicates that the subject should be treated with any of the compositions provided herein).

The term “pediatric patient” as used herein refers to a patient under the age of 16 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman—1996; Rudolph—2002; and Avery—1994.

In some embodiments of any of the methods or uses described herein, an assay is used to determine whether the patient has KRAS G12C mutation and/or STK11 genomic alteration using a sample (e.g., a biological sample or a biopsy sample (e.g., a paraffin-embedded biopsy sample) from a patient (e.g., a patient suspected of having a KRAS G12C mutant lung cancer, a patient having one or more symptoms of a KRAS G12C mutant lung cancer, and/or a patient that has an increased risk of developing a KRAS G12C mutant lung cancer) can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof.

The term “regulatory agency” is a country's agency for the approval of the medical use of pharmaceutical agents with the country. For example, a non-limiting example of a regulatory agency is the U.S. Food and Drug Administration (FDA).

As used herein, a “therapeutically effective amount” of a compound is an amount that is sufficient to ameliorate, or in some manner reduce a symptom or stop or reverse progression of a condition, or negatively modulate or inhibit the activity of KRas G12C. Such amount may be administered as a single dosage or may be administered according to a regimen, whereby it is effective.

As used herein, “treatment” means any manner in which the symptoms or pathology of a condition, disorder or disease are ameliorated or otherwise beneficially altered. Treatment also encompasses any pharmaceutical use of the compositions herein.

As used herein, “amelioration of the symptoms” of a particular disorder by administration of a particular pharmaceutical composition refers to any lessening, whether permanent or temporary, lasting or transient that can be attributed to or associated with administration of the composition.

As used herein, the term “about” when used to modify a numerically defined parameter (e.g., the dose of adagrasib or a pharmaceutically acceptable salt thereof, or the length of treatment time described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term “pharmaceutically acceptable salt” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. In one embodiment, a dose of the active compound for all of the above-mentioned conditions is in the range from about 0.01 to 300 mg/kg, for example 0.1 to 100 mg/kg per day, and as a further example 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising adagrasib may be used in the methods of use described herein.

### ILLUSTRATIVE EMBODIMENTS

The invention is based, in part, on the discovery that KRAS G12C mutant lung cancers (e.g., NSCLC) that contain one or more mutations in STK11, such as a loss-of-function mutation, may be particularly susceptible to treatment with adagrasib (MRTX849). In some embodiments adagrasib is administered to a mammalian subject, such as a human patient. Since the STK11 is often mutated in NSCLC, this therapeutic approach may be particularly useful for the treatment of NSCLC.

### Biomarkers

In certain embodiments, the present disclosure concerns the detection of a mutation or expression of genes, such as STK11. These genes can be used to predict response to adagrasib such as for the treatment of cancer, specifically KRAS G12C mutant lung cancer. The STK11 genes may have a mutation that results in loss of wild-type function or loss of expression, such as through non-mutational mechanism including genomic loss or promoter methylation. The loss of wild-type serine/threonine-protein kinase STK11 (LKB1) protein function can sensitize KRAS G12C mutant cells to adagrasib.

STK11, also named liver kinase B1 (LKB1), whose germline inactivation is responsible of the autosomal dominant Peutz-Jeghers syndrome, is a tumor-suppressor gene frequently mutated in NSCLC. The STK11 gene encodes for the serine/threonine-protein kinase STK11 protein, which controls the activity of AMP-activated protein kinase (AMPK) family members, thereby playing a role in various processes such as cell metabolism, cell polarity, apoptosis and DNA damage response. Serine/threonine-protein kinase STK11 acts by phosphorylating the T-loop of AMPK family proteins, thus promoting their activity. The STK11 gene is the second most commonly mutated tumor suppressor gene in NSCLC. STK11 loss-of-function mutations or loss of serine/threonine-protein kinase STK11 expression (often through non-mutational mechanisms like genomic loss or promoter methylation) occur more frequently in NSCLC than alterations in other genes such as EGFR, ALK, ROS, RET and BRAF combined.

The present methods can comprise detecting somatic mutations, loss of heterozygosity, whole gene deletions, decreased expression, or DNA methylation in the promoter region of STK11. In some embodiments, mutations in STK11 may arise in a variety of sites in a cancer.

The gene mutation or expression may be analyzed from a patient sample. The patient sample can be any bodily tissue or fluid that includes nucleic acids from the lung cancer in the subject. In certain embodiments, the sample will be a blood sample comprising circulating tumor cells or cell free DNA. In other embodiments, the sample can be a tissue, such as a lung tissue. The lung tissue can be from a tumor tissue and may be fresh frozen or formalin-fixed, paraffin-embedded (FFPE). In certain embodiments, a lung tumor FFPE sample is obtained.

Samples that are suitable for use in the methods described herein contain genetic material, e.g., genomic DNA (gDNA). Genomic DNA is typically extracted from biological samples such as blood or mucosal scrapings of the lining of the mouth, but can be extracted from other biological samples including urine, tumor, or expectorant. The sample itself will typically include nucleated cells (e.g., blood or buccal cells) or tissue removed from the subject including normal or tumor tissue. Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

In some cases, a biological sample may be processed for DNA isolation. For example, DNA in a cell or tissue sample can be separated from other components of the sample.

Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. See, e.g., Ausubel—2003. The sample can be concentrated and/or purified to isolate DNA. All samples obtained from a subject, including those subjected to any sort of further processing, are considered to be obtained from the subject. Routine methods can be used to extract genomic DNA from a biological sample, including, for example, phenol extraction. Alternatively, genomic DNA can be extracted with kits such as the QIAamp® Tissue Kit (Qiagen, Chatsworth, Calif.) and the Wizard® Genomic DNA purification kit (Promega). Non-limiting examples of sources of samples include urine, blood, and tissue. The biological sample may comprise or consist of cancerous cells or a tumor.

The presence or absence of mutations as described herein can be determined using methods known in the art. For example, gel electrophoresis, capillary electrophoresis, size exclusion chromatography, sequencing, and/or arrays can be used to detect the presence or absence of mutations. Amplification of nucleic acids, where desirable, can be accomplished using methods known in the art, e.g., PCR. In one example, a sample (e.g., a sample comprising genomic DNA), is obtained from a subject. The DNA in the sample is then examined to determine the identity of a mutation as described herein. A mutation can be detected by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular variant.

A set of probes typically refers to a set of primers, usually primer pairs, and/or detectably-labeled probes that are used to detect the target genetic variations used in the actionable treatment recommendations of the present disclosure. The primer pairs are used in an amplification reaction to define an amplicon that spans a region for a target genetic variation for each of the aforementioned genes. The set of amplicons are detected by a set of matched probes. In an exemplary embodiment, the present methods may use TaqMan™ (Roche Molecular Systems, Pleasanton, Calif.) assays that are used to detect a set of target genetic variations. In one embodiment, the set of probes are a set of primers used to generate amplicons that are detected by a nucleic acid sequencing reaction, such as a next generation sequencing reaction. In these embodiments, for example, AmpliSEQ™ (Life Technologies/Ion Torrent, Carlsbad, Calif.) or TruSEQ™ (Illumina, San Diego, Calif.) technology can be employed.

Analysis of nucleic acid markers can be performed using techniques known in the art including, without limitation, sequence analysis, and electrophoretic analysis. Non limiting examples of sequence analysis include Maxam-Gilbert sequencing, Sanger sequencing, capillary array DNA sequencing, thermal cycle sequencing (Sears—1992), solid-phase sequencing (Zimmerman—1992), sequencing with mass spectrometry such as matrix-assisted laser desorption/ionization time-of-flight mass spectrometry (MALDI-TOF/MS; Fu—1998, 1998), and sequencing by hybridization (Chee—1996; Drmanac—1993; Drmanac—1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

Other methods of nucleic acid analysis can include direct manual sequencing (Church—1988; Sanger—1977; U.S. Pat. No. 5,288,644); automated fluorescent sequencing; single-stranded conformation polymorphism assays (SSCP) (Schafer—1995); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield—1989); denaturing high performance liquid chromatography (DHPLC, Underhill—1997); infrared matrix-assisted laser desorption/ionization (IR-MALDI) mass spectrometry (WO 99/57318); mobility shift analysis; restriction enzyme analysis (Flavell—1978; Geever—1981); quantitative real-time PCR (Raca—2004); heteroduplex analysis; chemical mismatch cleavage (CMC) (Cotton—1985); RNase protection assays (Myers—1985); use of polypeptides that recognize nucleotide mismatches, e.g., E. coli mutS protein; allele-specific PCR, and combinations of such methods. See, e.g., U.S. Patent Publication No. 2004/0014095, which is incorporated herein by reference in its entirety.

In one example, a method of identifying a mutation in a sample comprises contacting a nucleic acid from said sample with a nucleic acid probe that is capable of specifically hybridizing to nucleic acid encoding a mutated protein, or fragment thereof incorporating a mutation, and detecting said hybridization. In a particular embodiment, said probe is detectably labeled such as with a radioisotope (3H, 32P, or 33P), a fluorescent agent (rhodamine, or fluorescein) or a chromogenic agent. In a particular embodiment, the probe is an antisense oligomer, for example PNA, morpholino-phosphoramidates, LNA or 2′-alkoxyalkoxy. The probe may be from about 8 nucleotides to about 100 nucleotides, or about 10 to about 75, or about 15 to about 50, or about 20 to about 30. In another aspect, said probes of the present disclosure are provided in a kit for identifying mutations in a sample, said kit comprising an oligonucleotide that specifically hybridizes to or adjacent to a site of mutation in the STK11 gene. The kit may further comprise instructions for treating patients having tumors that contain STK11 mutations with a DDR inhibitor based on the result of a hybridization test using the kit.

In another aspect, a method for detecting STK11 mutation in a sample comprises amplifying from said sample nucleic acids corresponding to STK11 or a fragment thereof suspected of containing a mutation, and comparing the electrophoretic mobility of the amplified nucleic acid to the electrophoretic mobility of corresponding wild-type STK11 gene or fragment thereof. A difference in the mobility indicates the presence of a mutation in the amplified nucleic acid sequence. Electrophoretic mobility may be determined on polyacrylamide gel.

Alternatively, nucleic acids may be analyzed for detection of mutations using Enzymatic Mutation Detection (EMD) (Del Tito—1998). EMD uses the bacteriophage resolvase T4 endonuclease VII, which scans along double-stranded DNA until it detects and cleaves structural distortions caused by base pair mismatches resulting from point mutations, insertions and deletions. Detection of two short fragments formed by resolvase cleavage, for example by gel electrophoresis, indicates the presence of a mutation. Benefits of the EMD method are a single protocol to identify point mutations, deletions, and insertions assayed directly from PCR reactions eliminating the need for sample purification, shortening the hybridization time, and increasing the signal-to-noise ratio. Mixed samples containing up to a 20-fold excess of normal DNA and fragments up to 4 kb in size can been assayed. However, EMD scanning does not identify particular base changes that occur in mutation positive samples requiring additional sequencing procedures to identity of the mutation if necessary. CEL I enzyme can be used similarly to resolvase T4 endonuclease VII as demonstrated in U.S. Pat. No. 5,869,245.

### Methods of Treatment

The invention provides methods for treating or delaying progression of KRas G12C-associated lung cancer, in particular, non-small cell lung cancer (NSCLC), with an STK11 genomic alteration (i.e., mutation) in a subject comprising administering to the subject a therapeutically effective amount of adagrasib, optionally in combination with a second anti-cancer therapy. The cancer in the subject may have more than one STK11 mutations.

In one embodiment, the subject is a human.

In one embodiment, the human is an adult patient.

In another embodiment, the human is a pediatric patient.

In one embodiment, the therapeutically effective amount of adagrasib, or a pharmaceutically acceptable salt thereof, is between about 200 and 1200 mg twice per day (BID).

In one embodiment, the therapeutically effective amount of adagrasib, or a pharmaceutically acceptable salt thereof, is about 600 mg twice per day (BID).

### Combination Therapies

In some embodiments of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

In one embodiment, the invention further comprises administering to the subject a second anti-cancer therapy. The second anti-cancer therapy may be selected from the group consisting of a surgery, an immunotherapy, a radiotherapy, a gene therapy, and a chemotherapy.

In one embodiment, the second anti-cancer therapy is an immune checkpoint inhibitor.

An immune checkpoint inhibitor is a drug that blocks proteins called checkpoints that are made by some types of immune system cells, such as T cells, and some cancer cells. These checkpoints help keep immune responses from being too strong and sometimes can keep T cells from killing cancer cells. When these checkpoints are blocked, T cells can kill cancer cells better. Examples of checkpoint proteins found on T cells or cancer cells include PD-1/PD-L1 and CTLA-4/B7-1/B7-2. Non-limiting examples of immune checkpoint inhibitors include ipilimumab, nivolumab, pembrolizumab, atezolizumab, avelumab, durvalumab, and cemiplimab.

In one embodiment, the second anti-cancer therapy is a PD-1/PD-L1 inhibitor.

In one embodiment, the PD-1/PD-L1 inhibitor is a PD-1 inhibitor.

In one embodiment, the PD-1 inhibitor is nivolumab, pembrolizumab, cemiplimab or tislelizumab, or a biosimilar thereof.

In another embodiment, the PD-1/PD-L1 inhibitor is a PD-L1 inhibitor.

In one embodiment, the PD-L1 inhibitor is atezolizumab, avelumab, or durvalumab, or a biosimilar thereof.

In one embodiment, the PD-1/PD-L1 inhibitor and the KRAS G12C inhibitor are administered on the same day.

In another embodiment, the PD-1/PD-L1 inhibitor and the KRAS G12C inhibitor are administered on different days.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few hours apart, or a few days apart.

An anti-cancer first treatment may be administered before, during, after, or in various combinations relative to a second anti-cancer treatment. The administrations may be in intervals ranging from concurrently to minutes to days to weeks. In embodiments where the first treatment is provided to a patient separately from the second treatment, one would generally ensure that a significant period of time did not expire between the time of each delivery, such that the two compounds would still be able to exert an advantageously combined effect on the patient. In such instances, it is contemplated that one may provide a patient with the first therapy and the second therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

### Parenteral Compositions and Formulations

In further embodiments, adagrasib (and, optionally, other active agents) may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,613,308, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

In some embodiments, adagrasib is administered orally to a subject.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

The following Examples are intended to illustrate further certain embodiments of the invention and are not intended to limit the scope of the invention.

### EXAMPLES

### Example 1

Association of STK11 mutation and clinical response to adagrasib in KRAS G12C mutant NSCLC

The effect of STK11 loss-of-function mutations on clinical response to adagrasib, a direct inhibitor of KRas G12C, was tested in a subset of patients with KRAS G12C mutant NSCLC who were enrolled on a Phase 1/2, multi-cohort trial of adagrasib in patients with KRAS G12C mutant advanced solid tumors (Mirati Study 849-001).

Patients could enroll onto Mirati Study 849-001 if a KRAS G12C mutation was detected in an archival tumor tissue specimen either by a Sponsor-approved local genotyping test or a central genotyping assay. Patients that were included in this analysis must have had a diagnosis of NSCLC, must have initiated adagrasib at the recommended Phase 2 dose of 600 mg BID, had KRAS G12C detected in tumor tissue for enrollment onto the trial, and had at least one on-treatment disease assessment evaluable for response.

Based on these criteria, patients were included from several cohorts in Mirati Study 849-001, including Phase 1 dose escalation, Phase 1b dose expansion, and Cohort A of Phase 2. Objective response was defined using RECIST 1.1 criteria (Eisenhauer—2009). Patients with objective response were classified as having either a complete response (CR), partial response (PR), unconfirmed CR (uCR) or unconfirmed PR (uPR). Patients without an objective response were designated as having either stable disease (SD) or progressive disease (PD) or not evaluable (NE) by RECIST 1.1 criteria or “SD+6WK”, which was defined as SD by RECIST 1.1 criteria that was durable for at least 6 weeks after the first SD assessment. To be evaluable for response, a patient must have had at least one on-study disease assessment prior to discontinuation or death or discontinue study treatment due to global deterioration of health status (i.e., clinical disease progression). Patients who discontinue treatment for other reasons, such as adverse events or withdrawal of consent, prior to the first on-study disease assessment were not included in the evaluable population.

All patients in this analysis had KRAS G12C and STK11 mutation status in baseline tumor samples determined by Sponsor-approved local next-generation sequencing (NGS)-based genotyping tests. All of the Sponsor-approved local genotyping tests were clinical assays intended for use in the management of cancer patients and were performed in academic medical center or commercial Clinical Laboratory Improvement Amendments (CLIA)-certified laboratories. STK11 mutations were designated as loss-of-function (i.e. pathogenic and “positive”) if the type of genetic alteration was as defined herein.

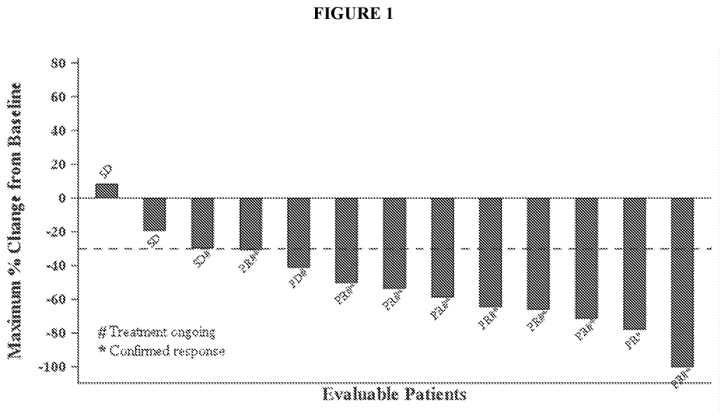

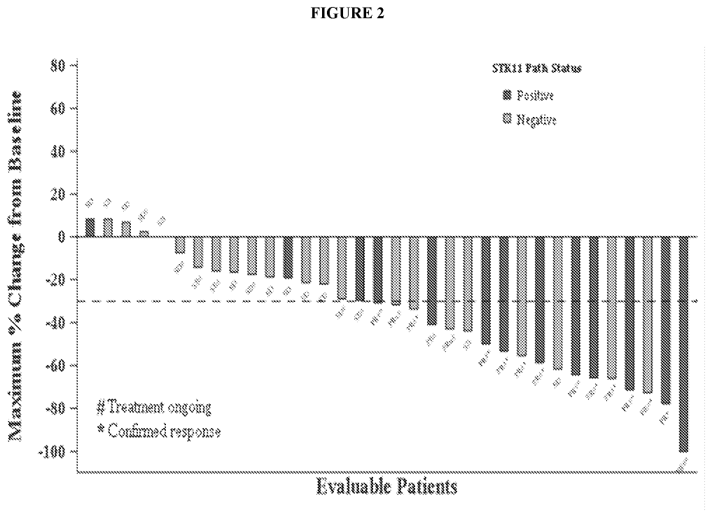

A total of 34 patients were included, of which 13 (38%) had STK11 loss-of-function mutations. The overall response rate, regardless of STK11 mutation status, was 41% (12 PRs and 2 uPRs in 34 evaluable patients) (Table 1). No patient had a CR or uCR. In patients with STK11 mutations, the response rate was 69% (9 PRs in 13 evaluable patients). In patients without STK11 mutations, the response was 24% (3 PRs and 2 uPRs in 21 evaluable patients). The difference in response rate between patients with and without STK11 mutations achieved statistical significance (P=0.0137, two-tailed Fisher exact test). Using RECIST 1.1 criteria, which requires confirmation disease assessments for designation as CR or PR, there was a statistically significant difference in ORR between patients with and without STK11 mutations (9 of 13 patients with PR, or 69% vs. 3 of 21 patients with PR, or 14%; P=0.0024, two-tailed Fisher exact test) (Table 1). Among patients with STK11 mutation in this cohort, regression of target lesions (any decrease in target lesion size from baseline) was observed in 12 of 13 (92%) patients (FIG. 1). In patients with wild-type STK11 in this cohort, regression of target lesions was observed in 17 of 21 (81%) patients (FIG. 2).

TABLE 1
Best Overall Response by STK11 mutation status in KRAS
G12C mutant NSCLC patients treated with adagrasib 600
mg BID monotherapy in Phase 1/1b and in the Phase 2
cohort with KRAS G12C identified in tumor tissue.

| STK11 | |
|---|---|
| Negative | Positive |

| Best Overall Response | N | % | N | % |
|---|---|---|---|---|
| PR | 3 | 14.29 | 9 | 69.23 |
| uPR | 2 | 9.52 | . | . |
| SD | 5 | 23.81 | 1 | 7.69 |
| SD + 6WK | 11 | 52.38 | 2 | 15.38 |
| PD | . | . | 1 | 7.69 |
| Total | 21 | 100.00 | 13 | 100.00 |

Patients with NSCLC harboring STK11 mutations have a poor prognosis. Furthermore, patients with NSCLC that harbor mutations in both KRAS and STK11 appear to have a particularly poor prognosis and their cancer is resistant to PD-1/PD-L1 inhibitors, which are standard-of-care for treatment of NSCLC patients. Patients with KRAS G12C and STK11 co-mutant NSCLC are in critical need of more effective therapies. Preliminary data presented here indicate that in NSCLC patients with KRAS G12C mutant identified in tumor tissue, STK11 loss-of-function mutations are associated with a higher rate of radiographic response with adagrasib treatment compared to patients with a wild-type STK11 gene. The mechanism for the apparent improved clinical activity of a direct inhibitor of KRas G12C in NSCLC patients with KRAS G12C and STK11 co-mutations compared to patients with NSCLC with KRAS G12C mutation alone remains to be determined. Nevertheless, adagrasib potentially represents a novel, effective treatment option for patients with KRAS G12C and STK11 co-mutant NSCLC.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

### Appendix 1

### Examples of STK11 Loss-of-Function Mutations

| Protein Change | Mutation Type | HGVS genomic nomenclature |
|---|---|---|
| F354L | Missense_Mutation | 19:g.1223125C > G |
| P281Rfs*6 | Frameshift_Deletion | 19:g.1221314del |
| P281Rfs*6 | Frameshift_Deletion | 19:g.1221314del |
| P281Rfs*6 | Frameshift_Deletion | 19:g.1221314del |
| P281Rfs*6 | Frameshift_Deletion | 19:g.1221314del |
| P281Rfs*6 | Frameshift_Deletion | 19:g.1221314del |
| P281Rfs*6 | Frameshift_Deletion | 19:g.1221314del |
| E130* | Nonsense_Mutation | 19:g.1219336G > T |
| E130* | Nonsense_Mutation | 19:g.1219336G > T |
| E120* | Nonsense_Mutation | 19:g.1218483G > T |
| Q37* | Nonsense_Mutation | 19:g.1207021C > T |
| Q37* | Nonsense_Mutation | 19:g.1207021C > T |
| Q37* | Nonsense_Mutation | 19:g.1207021C > T |
| Q37* | Nonsense_Mutation | 19:g.1207021C > T |
| Q37* | Nonsense_Mutation | 19:g.1207021C > T |
| Y60* | Frameshift_Deletion | 19:g.1207092del |
| Q159* | Nonsense_Mutation | 19:g.1220382C > T |
| Q159* | Nonsense_Mutation | 19:g.1220382C > T |
| Q159* | Nonsense_Mutation | 19:g.1220382C > T |
| Y60* | Nonsense_Mutation | 19:g.1207092C > A |
| E165* | Nonsense_Mutation | 19:g.1220400G > T |
| E165* | Nonsense_Mutation | 19:g.1220400G > T |
| E165* | Nonsense_Mutation | 19:g.1220400G > T |
| E165* | Nonsense_Mutation | 19:g.1220400G > T |
| E165* | Nonsense_Mutation | 19:g.1220400G > T |
| K191* | Nonsense_Mutation | 19:g.1220478A > T |
| K191* | Nonsense_Mutation | 19:g.1220478A > T |
| Q170* | Nonsense_Mutation | 19:g.1220415C > T |
| Q220* | Nonsense_Mutation | 19:g.1220640C > T |
| Q220* | Nonsense_Mutation | 19:g.1220640C > T |
| Q220* | Nonsense_Mutation | 19:g.1220640C > T |
| Q220* | Nonsense_Mutation | 19:g.1220640C > T |
| Y60* | Nonsense_Mutation | 19:g.1207090_1207091insA |
| Y60* | Nonsense_Mutation | 19:g.1207090_1207091insA |
| D53Gfs*110 | Frameshift_Insertion | 19:g.1207064_1207065insG |
| D53Gfs*110 | Frameshift_Insertion | 19:g.1207064_1207065insG |
| D53Gfs*110 | Frameshift_Insertion | 19:g.1207064_1207065insG |
| E223K | Missense_Mutation | 19:g.1220649G > A |
| Y272* | Nonsense_Mutation | 19:g.1221293C > A |
| W332* | Nonsense_Mutation | 19:g.1223059G > A |
| E57Kfs*7 | Frameshift_Deletion | 19:g.1207077del |
| E57Kfs*7 | Frameshift_Deletion | 19:g.1207077del |
| K84* | Nonsense_Mutation | 19:g.1207162A > T |
| K84* | Nonsense_Mutation | 19:g.1207162A > T |
| D53Tfs*11 | Frameshift_Deletion | 19:g.1207065del |
| D53Tfs*11 | Frameshift_Deletion | 19:g.1207065del |
| D53Tfs*11 | Frameshift_Deletion | 19:g.1207065del |
| E223* | Nonsense_Mutation | 19:g.1220649G > T |
| E223* | Nonsense_Mutation | 19:g.1220649G > T |
| E223* | Nonsense_Mutation | 19:g.1220649G > T |
| E223* | Nonsense_Mutation | 19:g.1220649G > T |
| E223* | Nonsense_Mutation | 19:g.1220649G > T |
| E223* | Nonsense_Mutation | 19:g.1220649G > T |
| E223* | Nonsense_Mutation | 19:g.1220649G > T |
| E223* | Nonsense_Mutation | 19:g.1220649G > T |
| E223* | Nonsense_Mutation | 19:g.1220649G > T |
| E223* | Nonsense_Mutation | 19:g.1220649G > T |
| E223* | Nonsense_Mutation | 19:g.1220649G > T |
| E33* | Nonsense_Mutation | 19:g.1207009G > T |
| Q123* | Nonsense_Mutation | 19:g.1218492C > T |
| E70* | Nonsense_Mutation | 19:g.1207120G > T |
| L282Afs*3 | Frameshift_Insertion | 19:g.1221313_1221314insC |
| L282Afs*3 | Frameshift_Insertion | 19:g.1221313_1221314insC |
| D176Tfs*111 | Frameshift_Deletion | 19:g.1220432del |
| K108* | Nonsense_Mutation | 19:g.1218447A > T |
| E65* | Nonsense_Mutation | 19:g.1207105G > T |
| K146* | Nonsense_Mutation | 19:g.1219384A > T |
| S69* | Nonsense_Mutation | 19:g.1207118C > A |
| S69* | Nonsense_Mutation | 19:g.1207118C > A |
| Y60* | Nonsense_Mutation | 19:g.1207092C > G |
| Y60* | Nonsense_Mutation | 19:g.1207092C > G |
| V243Sfs*44 | Frameshift_Deletion | 19:g.1220706del |
| Q100* | Nonsense_Mutation | 19:g.1218423C > T |
| Q100* | Nonsense_Mutation | 19:g.1218423C > T |
| Q100* | Nonsense_Mutation | 19:g.1218423C > T |
| Q100Nfs*3 | Frameshift_Deletion | 19:g.1218423del |
| D176G | Missense_Mutation | 19:g.1220434A > G |
| G163S | Missense_Mutation | 19:g.1220394G > A |
| Y246* | Nonsense_Mutation | 19:g.1221215C > G |
| D343Gfs*17 | Frameshift_Insertion | 19:g.1223087_1223088insA |
| Q100Pfs*51 | Frameshift_Deletion | 19:g.1218422_1218456del |
| K44Tfs*2 | Frameshift_Deletion | 19:g.1207042_1207057del |
| E57Gfs*106 | Frameshift_Insertion | 19:g.1207076_1207077insG |
| H202Pfs*64 | Frameshift_Insertion | 19:g.1220585_1220587delinsTCCC |
| G188Afs*99 | Frameshift_Deletion | 19:g.1220469del |
| G188Afs*99 | Frameshift_Deletion | 19:g.1220469del |
| Y131* | Nonsense_Mutation | 19:g.1219341C > A |
| Y131* | Nonsense_Mutation | 19:g.1219341C > A |
| E120Kfs*9 | Frameshift_Deletion | 19:g.1218483del |
| X285_splice | Splice-Site | 19:g.1221332_1221361del |
| P314Lfs*22 | Frameshift_Deletion | 19:g.1223003del |
| P314Lfs*22 | Frameshift_Deletion | 19:g.1223003del |
| K329* | Nonsense_Mutation | 19:g.1223048A > T |
| E57* | Nonsense_Mutation | 19:g.1207081G > T |
| L282Sfs*5 | Frameshift_Deletion | 19:g.1221320del |
| L282Sfs*5 | Frameshift_Deletion | 19:g.1221320del |
| Q214* | Nonsense_Mutation | 19:g.1220622C > T |
| Q214* | Nonsense_Mutation | 19:g.1220622C > T |
| G163R | Missense_Mutation | 19:g.1220394G > C |
| W332Cfs*4 | Frameshift_Deletion | 19:g.1223058del |
| Y60Sfs*4 | Frameshift_Deletion | 19:g.1207091del |
| K311Nfs*4 | Frameshift_Deletion | 19:g.1222996_1223003del |
| G163Afs*124 | Frameshift_Deletion | 19:g.1220394del |
| G163Afs*124 | Frameshift_Deletion | 19:g.1220394del |
| K78* | Nonsense_Mutation | 19:g.1207144A > T |
| K96* | Nonsense_Mutation | 19:g.1207198A > T |
| L85* | Nonsense_Mutation | 19:g.1207166T > A |
| L85* | Nonsense_Mutation | 19:g.1207166T > A |
| L85* | Nonsense_Mutation | 19:g.1207166T > A |
| P275Gfs*9 | Frameshift_Deletion | 19:g.1221299_1221300del |
| D208Qfs*53 | Frameshift_Deletion | 19:g.1220599_1220612del |
| F204Sfs*83 | Frameshift_Deletion | 19:g.1220592del |
| G279Afs*8 | Frameshift_Deletion | 19:g.1221312del |
| Y36* | Nonsense_Mutation | 19:g.1207020C > G |
| K97* | Nonsense_Mutation | 19:g.1207201A > T |
| 1300Gfs*2 | Frameshift_Deletion | 19:g.1221983_1222003delinsGGAT |
| C132Wfs*29 | Frameshift_Deletion | 19:g.1219344del |
| R40Afs*11 | Frameshift_Deletion | 19:g.1207029del |
| E291* | Nonsense_Mutation | 19:g.1221956G > T |
| E291* | Nonsense_Mutation | 19:g.1221956G > T |
| F264Rfs*22 | Frameshift_Deletion | 19:g.1221264_1221267del |
| A43Pfs*8 | Frameshift_Deletion | 19:g.1207037del |
| K64* | Nonsense_Mutation | 19:g.1207102A > T |
| Y36 K48delins* | Nonsense_Mutation | 19:g.1207019_1207054del |
| S283* | Frameshift_Deletion | 19:g.1221321_1221322del |
| W239* | Nonsense_Mutation | 19:g.1220699G > A |
| Q305* | Nonsense_Mutation | 19:g.1221998C > T |
| G268Vfs*9 | Frameshift_Deletion | 19:g.1221279_1221309del |
| G135Afs*23 | Frameshift_Deletion | 19:g.1219350_1219363del |
| Y118* | Nonsense_Mutation | 19:g.1218479C > G |
| K64Rfs*32 | Frameshift_Deletion | 19:g.1207102del |
| N226Rfs*39 | Frameshift_Deletion | 19:g.1220659_1220660del |
| M136Nfs*24 | Frameshift_Deletion | 19:g.1219351_1219358del |
| P217Rfs*70 | Frameshift_Deletion | 19:g.1220629del |
| A198Pfs*89 | Frameshift_Deletion | 19:g.1220498del |
| Q152Rfs*9 | Frameshift_Deletion | 19:g.1219401del |
| Q152Rfs*9 | Frameshift_Deletion | 19:g.1219401del |
| V66Sfs*98 | Frameshift_Insertion | 19:g.1207108_1207109delinsAGCACC |
| E120 E121delinsK* | Nonsense_Mutation | 19:g.1218483_1218486delinsAAAT |
| W308* | Nonsense_Mutation | 19:g.1222987G > A |
| A76Gfs*21 | Frameshift_Insertion | 19:g.1207135_1207136insGG |
| E293* | Nonsense_Mutation | 19:g.1221962G > T |
| K44* | Nonsense_Mutation | 19:g.1207042A > T |
| L117lfs*45 | Frameshift_Deletion | 19:g.1218470_1218471del |
| D162_G163delinsYF | Missense_Mutation | 19:g.1220391_1220395delinsTACTT |
| N181Tfs*106 | Frameshift_Deletion | 19:g.1220444del |
| S240* | Nonsense_Mutation | 19:g.1220701C > A |
| L50Rfs*12 | Frameshift_Deletion | 19:g.1207061_1207067del |
| K78N | Missense_Mutation | 19:g.1207146G > T |
| R42Lfs*9 | Frameshift_Deletion | 19:g.1207036_1207038delinsTT |
| V243Gfs*23 | Frameshift_Insertion | 19:g.1220705_1220706insG |
| V243Gfs*23 | Frameshift_Insertion | 19:g.1220705_1220706insG |
| F255lfs*11 | Frameshift_Insertion | 19:g.1221239_1221240insA |
| X199_splice | Splice-Site | 19:g.1220504_1220544del |
| X148_splice | Splice-Site | 19:g.1219386_1219414del |
| A347Rfs*9 | Frameshift_Deletion | 19:g.1223099_1223109del |
| D327Vfs*28 | Frameshift_Deletion | 19:g.1223039_1223052del |
| E256* | Nonsense_Mutation | 19:g.1221243G > T |
| E256* | Nonsense_Mutation | 19:g.1221243G > T |
| E256* | Nonsense_Mutation | 19:g.1221243G > T |
| P221Afs*61 | Frameshift_Deletion | 19:g.1220643_1220662delinsGCGC |
| X171_splice | Splice-Site | 19:g.1220416_1220760del |
| V197Rfs*69 | Frameshift_Insertion | 19:g.1220491_1220492insG |
| Y36Sfs*125 | Frameshift_Deletion | 19:g.1207019_1207023del |
| D23Sfs*25 | Frameshift_Deletion | 19:g.1206979_1206988del |
| E223V | Missense_Mutation | 19:g.1220650A > T |
| R39Pfs*8 | Frameshift_Deletion | 19:g.1207026_1207038del |
| T249Gfs*37 | Frameshift_Deletion | 19:g.1221222_1221226delinsG |
| A206Rfs*81 | Frameshift_Deletion | 19:g.1220597del |
| A206Rfs*81 | Frameshift_Deletion | 19:g.1220597del |
| E265* | Frameshift_Insertion | 19:g.1221266_1221267insT |
| G279Rfs*6 | Frameshift_Deletion | 19:g.1221311_1221317del |
| G279Vfs*8 | Frameshift_Deletion | 19:g.1221313_1221314delinsT |
| S216Rfs*43 | Frameshift_Deletion | 19:g.1220627_1220646del |
| X199_splice | Splice-Site | 19:g.1220506T > C |
| D229Tfs*58 | Frameshift_Deletion | 19:g.1220666del |
| N90Tfs*6 | Frameshift_Deletion | 19:g.1207176del |
| P281Gfs*3 | Frameshift_Deletion | 19:g.1221318_1221321delinsGG |
| R211Lfs*76 | Frameshift_Deletion | 19:g.1220613_1220615delinsTT |
| G196Afs*91 | Frameshift_Deletion | 19:g.1220492del |
| M1? | Translation_Start_Site | 19:g.1206915G > A |
| L201Pfs*63 | Frameshift_Deletion | 19:g.1220580_1220584del |
| E145* | Frameshift_Insertion | 19:g.1219380_1219381insT |
| P280Lfs*7 | Frameshift_Deletion | 19:g.1221316_1221317delinsT |
| 1300Afs*12 | Frameshift_Deletion | 19:g.1221983_1221999del |
| A218Gfs*48 | Frameshift_Insertion | 19:g.1220628_1220629insC |
| Y60* | Nonsense_Mutation | 19:g.1207090_1207091insAG |
| F148Sfs*13 | Frameshift_Deletion | 19:g.1219389del |
| Y253* | Nonsense_Mutation | 19:g.1221236C > A |
| STK11-intragenic | Fusion | |
| STK11-intragenic | Fusion | |
| STK11-intragenic | Fusion | |
| STK11-intragenic | Fusion | |
| STK11-intragenic | Fusion | |
| STK11-intragenic | Fusion | |
| STK11-intragenic | Fusion | |
| STK11-intragenic | Fusion | |
| STK11-intragenic | Fusion | |
| STK11-intragenic | Fusion | |
| STK11-intragenic | Fusion | |
| MIDN-STK11 fusion | Fusion | |
| MIDN-STK11 fusion | Fusion | |
| STK11-SBNO2 fusion | Fusion | |
| STK11-HMHA1 | Fusion | |
| fusion | | |
| C19orf26-STK11 | Fusion | |
| fusion | | |
| C19orf26-STK11 | Fusion | |
| fusion | | |
| REEP6-STK11 fusion | Fusion | |
| CACNA1A-STK11 | Fusion | |
| fusion | | |
| X155_splice | Splice-Site | 19:g.1220370A > T |
| X155_splice | Splice-Site | 19:g.1220370A > T |
| X307_splice | Splice-Site | 19:g.1222006G > T |
| X307_splice | Splice-Site | 19:g.1222006G > T |
| X307_splice | Splice-Site | 19:g.1222983G > A |
| S216F | Missense_Mutation | 19:g.1220629C > T |
| S216F | Missense_Mutation | 19:g.1220629C > T |
| S216F | Missense_Mutation | 19:g.1220629C > T |
| S216F | Missense_Mutation | 19:g.1220629C > T |
| S216F | Missense_Mutation | 19:g.1220629C > T |
| S216F | Missense_Mutation | 19:g.1220629C > T |
| S216F | Missense_Mutation | 19:g.1220629C > T |
| S216F | Missense_Mutation | 19:g.1220629C > T |
| S216F | Missense_Mutation | 19:g.1220629C > T |
| D194N | Missense_Mutation | 19:g.1220487G > A |
| D194N | Missense_Mutation | 19:g.1220487G > A |
| X155_splice | Splice-Site | 19:g.1220371G > T |
| X155_splice | Splice-Site | 19:g.1220371G > T |
| X155_splice | Splice-Site | 19:g.1220371G > T |
| D194Y | Missense_Mutation | 19:g.1220487G > T |
| D194Y | Missense_Mutation | 19:g.1220487G > T |
| D194Y | Missense_Mutation | 19:g.1220487G > T |
| D194Y | Missense_Mutation | 19:g.1220487G > T |
| X245_splice | Splice-Site | 19:g.1220717G > T |
| X245_splice | Splice-Site | 19:g.1220717G > T |
| X245_splice | Splice-Site | 19:g.1220717G > T |
| X245_splice | Splice-Site | 19:g.1220717G > T |
| X97_splice | Splice-Site | 19:g.1218415G > T |
| W308L | Missense_Mutation | 19:g.1222986G > T |
| W308L | Missense_Mutation | 19:g.1222986G > T |
| W308L | Missense_Mutation | 19:g.1222986G > T |
| X200_splice | Splice-Site | 19:g.1220578A > G |
| X155_splice | Splice-Site | 19:g.1219413G > T |
| W308C | Missense_Mutation | 19:g.1222987G > T |
| W308C | Missense_Mutation | 19:g.1222987G > T |
| X288_splice | Splice-Site | 19:g.1221946A > C |
| X245_splice | Splice-Site | 19:g.1220717G > A |
| X245_splice | Splice-Site | 19:g.1220717G > A |
| X307_splice | Splice-Site | 19:g.1222982A > G |
| X155_splice | Splice-Site | 19:g.1220370A > G |
| X125_splice | Splice-Site | 19:g.1218500A > G |
| X125_splice | Splice-Site | 19:g.1218500A > G |
| X288_splice | Splice-Site | 19:g.1221947G > A |
| X97_splice | Splice-Site | 19:g.1207203G > A |
| X245_splice | Splice-Site | 19:g.1220718T > G |
| X155_splice | Splice-Site | 19:g.1219412_1219422del |
| X155_splice | Splice-Site | 19:g.1220371_1220372delinsTT |
| X307_splice | Splice-Site | 19:g.1222982A > T |
| X307_splice | Splice-Site | 19:g.1222982A > T |
| X155_splice | Splice-Site | 19:g.1219414T > A |
| X288_splice | Splice-Site | 19:g.1221947G > T |
| X97_splice | Splice-Site | 19:g.1207204T > A |
| X200_splice | Splice-Site | 19:g.1220578A > T |
| X200_splice | Splice-Site | 19:g.1220578A > T |
| X200_splice | Splice-Site | 19:g.1220574_1220607del |
| H174D | Missense_Mutation | 19:g.1220427C > G |
| X155_splice | Splice-Site | 19:g.1220370A > C |
| D194V | Missense_Mutation | 19:g.1220488A > T |
| D194V | Missense_Mutation | 19:g.1220488A > T |
| H174R | Missense_Mutation | 19:g.1220428A > G |
| X307_splice | Splice-Site | 19:g.1222983G > C |
| X307_splice | Splice-Site | 19:g.1222983G > T |
| X307_splice | Splice-Site | 19:g.1222983G > T |
| X155_splice | Splice-Site | 19:g.1219413G > C |
| X97_splice | Splice-Site | 19:g.1207201_1207203delinsGAT |
| X288_splice | Splice-Site | 19:g.1221340G > A |
| X288_splice | Splice-Site | 19:g.1221340G > A |
| H174L | Missense_Mutation | 19:g.1220428A > T |
| X125_splice | Splice-Site | 19:g.1219321A > T |
| X245_splice | Splice-Site | 19:g.1220717G > C |
| D194G | Missense_Mutation | 19:g.1220488A > G |
| X155_splice | Splice-Site | 19:g.1220201_1220383del |
| X125_splice | Splice-Site | 19:g.1219322C > T |
| X307_splice | Splice-Site | 19:g.1222611_1223004del |
| R304W | Missense_Mutation | 19:g.1221995C > T |
| R304W | Missense_Mutation | 19:g.1221995C > T |
| R304W | Missense_Mutation | 19:g.1221995C > T |
| R304W | Missense_Mutation | 19:g.1221995C > T |
| A205T | Missense_Mutation | 19:g.1220595G > A |
| G196V | Missense_Mutation | 19:g.1220494G > T |
| G196V | Missense_Mutation | 19:g.1220494G > T |
| G196V | Missense_Mutation | 19:g.1220494G > T |
| G242V | Missense_Mutation | 19:g.1220707G > T |
| G242V | Missense_Mutation | 19:g.1220707G > T |
| W239C | Missense_Mutation | 19:g.1220699G > T |
| W239C | Missense_Mutation | 19:g.1220699G > T |
| P221L | Missense_Mutation | 19:g.1220644C > T |
| P221L | Missense_Mutation | 19:g.1220644C > T |
| P221L | Missense_Mutation | 19:g.1220644C > T |
| P221L | Missense_Mutation | 19:g.1220644C > T |
| G251V | Missense_Mutation | 19:g.1221229G > T |
| G251V | Missense_Mutation | 19:g.1221229G > T |
| G242W | Missense_Mutation | 19:g.1220706G > T |
| G251C | Missense_Mutation | 19:g.1221228G > T |
| E165Q | Missense_Mutation | 19:g.1220400G > C |
| E165Q | Missense_Mutation | 19:g.1220400G > C |
| P179L | Missense_Mutation | 19:g.1220443C > T |
| P179L | Missense_Mutation | 19:g.1220443C > T |
| P179L | Missense_Mutation | 19:g.1220443C > T |
| P179R | Missense_Mutation | 19:g.1220443C > G |
| P179Q | Missense_Mutation | 19:g.1220443C > A |
| G196R | Missense_Mutation | 19:g.1220493G > C |
| N181S | Missense_Mutation | 19:g.1220449A > G |
| E165K | Missense_Mutation | 19:g.1220400G > A |
| N181T | Missense_Mutation | 19:g.1220449A > C |
| W239S | Missense_Mutation | 19:g.1220698G > C |
| G242R | Missense_Mutation | 19:g.1220706G > A |
| G242V | Missense_Mutation | 19:g.1220707_1220708delinsTT |
| P281L | Missense_Mutation | 19:g.1221319C > T |
| R409W | Missense_Mutation | 19:g.1226569C > T |

### REFERENCES

Alamgeer et al., (2013) Current Opin Pharmcol. 13:394-401.

Santos et al., (1984) Science 223:661-664.

Samatar and Poulikakos (2014) Nat Rev Drug Disc 13 (12):928-942 doi: 10.1038/nrd428.

Dogan et al., (2012) Clin Cancer Res. 18 (22):6169-6177, published online 2012 Sep 26. doi: 10.1158/1078-0432. CCR-11-3265.

McCormick (2015) Clin Cancer Res. 21 (8):1797-1801.

Ostrem et al., (2013) Nature 503:548-551.

Fell et al., (2020) J. Med. Chem. 63, 6679-6693.

Berhman R E, Kliegman R, Arvin A M, Nelson W E. Nelson Textbook of Pediatrics, 15th Ed. Philadelphia: W. B. Saunders Company, 1996.

Rudolph A M, et al. Rudolph's Pediatrics, 21st Ed. New York: McGraw-Hill, 2002.

Avery M D, First L R. Pediatric Medicine, 2nd Ed. Baltimore: Williams & Wilkins; 1994.

Fu J, et al. (1998) Repeated tertiary fold of RNA polymerase II and implications for DNA binding. J Mol Biol 280 (3):317-22.

Sanchez-Cespedes M, Parrella P, Esteller M, et al. Inactivation of LKB1/STK11 is a common event in adenocarcinomas of the lung. Cancer Res 2002; 62:3659-62.

Ji H, Ramsey M R, Hayes D N, et al. LKB1 modulates lung cancer differentiation and metastasis. Nature 2007; 448: 807-10.

Gurumurthy S, Xie S Z, Alagesan B, Kim J, Yusuf R Z, Saez B, et al. The Lkb 1 metabolic sensor maintains haematopoietic stem cell survival. Nature 2010; 468:659-63.

Gill R K, Yang S H, Meerzaman D et al. Frequent homozygous deletion of the LKB1/STK11 gene in non-small cell lung cancer. Oncogene 2011; 30:3784-3791.

Gao J, Aksoy B A, Dogrusoz U et al. Integrative analysis of complex cancer genomics and clinical profiles using the cBioPortal. Sci Signal 2013; 6:p11.

Cerami E, Gao J, Dogrusoz U et al. The cBio cancer genomics portal: An open platform for exploring multidimensional cancer genomics data. Cancer Discov 2012; 2:401-404.

Zehir A, et al. Mutational landscape of metastatic cancer revealed from prospective clinical sequencing of patients. Nat Med. 2017 June; 23 (6):703-713

Robinson D R, et al. Integrative clinical genomics of metastatic cancer. Nature. 2017 Aug. 17; 548 (7667):297-303

Sanchez-Vega F, Mina M, Armenia J et al. Oncogenic signaling pathways in The Cancer Genome Atlas. Cell 2018; 173:321.337.e10.

Hemminki A, Markie D, Tomlinson I, et al. (1998). A serine/threonine kinase gene defective in Peutz— Jeghers syndrome. Nature 18: 184-187.

Alessi, D. R., Sakamoto, K., and Bayascas, J. R. (2006). LKB1-dependent signaling pathways. Annu. Rev. Biochem. 75, 137-163.

Sanchez-Cespedes M. A role for LKB1 gene in human cancer beyond the Peutz-Jeghers syndrome. Oncogene (2007) 26, 7825-7832.

Richards S, et al. Standards and Guidelines for the Interpretation of Sequence Variants: A Joint Consensus Recommendation of the American College of Medical Genetics and Genomics and the Association for Molecular Pathology. Genet Med. 2015 May; 17 (5): 405-424.

Jenne D E, Reimann H, Nezu J, et al. (1998). Peutz-Jeghers syndrome is caused by mutations in a novel serine threonine kinase. Nat Genet 18: 38-44.

Alessi D R, Sakamoto K, Bayascas J R. (2006). LKB1-dependent signaling pathways. Annu Rev Biochem 137-163.

Chakravarty D, Gao J, Phillips S M et al. OncoKB: A precision oncology knowledge base.

JCO Precis Oncol 2017; 2017

Zeqiraj E, Filippi B M, Deak M et al. Structure of the LKB1-STRAD-MO25 complex reveals an allosteric mechanism of kinase activation. Science 2009; 326:1707-1711.

Laplante M, Sabatini D M. mTOR signaling at a glance. J Cell Sci 2009; 122:3589-3594.

Shackelford, D. B., and Shaw, R. J. (2009). The LKB1-AMPK pathway: Metabolism and growth control in tumour suppression. Nat. Rev. Cancer. 9, 563-75

Wodarz A, Nathke I. Cell polarity in development and cancer. Nat Cell Biol 2007; 9:1016-24.

Mihaylova M M, Shaw R I. The AMPK signalling pathway coordinates cell growth, autophagy and metabolism. Nat Cell Biol 2011; 13:1016-23.

Li F et al. LKB1 Inactivation Elicits a Redox Imbalance to Modulate Non-small Cell Lung Cancer Plasticity and Therapeutic Response. Cancer Cell 27, 698-711 (2015). [PubMed: 25936644]

Zhang H et al. Lkb1 inactivation drives lung cancer lineage switching governed by Polycomb Repressive Complex 2. Nat Commun 8, 14922 (2017). [PubMed: 28387316]

Kottakis F et al. LKB1 loss links serine metabolism to DNA methylation and tumorigenesis. Nature 539, 390-395 (2016). [PubMed: 27799657]

Liu Y et al. Metabolic and functional genomic studies identify deoxythymidylate kinase as a target in LKB1-mutant lung cancer. Cancer Discov 3, 870-9 (2013). [PubMed: 23715154]

Shakelford D B and Shaw R I. The LKB1-AMPK pathway: metabolism and growth control in tumor suppression. Nat Rev Cancer. 2009 August; 9 (8): 563-575

Launonen V. (2005). Mutations in the human LKB1/STK11 gene. Hum Mutat 26:291-297.

Esteve-Puig R, Gil R, Gonzalez-Sanchez E et al. A mouse model uncovers LKB1 as an UVB induced DNA damage sensor mediating CDKN1A (p21WAF1/CIP1) degradation. PLoS Genet 2014; 10:e1004721.

Skoulidis F and Heymach J V. Co-occurring genomic alterations in non-small cell lung cancer biology and therapy. Nat Rev Cancer. 2019 September; 19 (9): 495-509.

Koyama S et al. STK11/LKB1 Deficiency Promotes Neutrophil Recruitment and Proinflammatory Cytokine Production to Suppress T-cell Activity in the Lung Tumor Microenvironment. Cancer Res 76, 999-1008 (2016).

Kitajima S et al. Suppression of STING Associated with LKB1 Loss in KRAS-Driven Lung Cancer. Cancer Discov 9, 34-45 (2019). [PubMed: 30297358] This is the first report that LKB1 loss results in impaired innate immune sensing of cytosolic DNA due to epigenetic repression of STING.

Skoulidis F et al. Co-occurring genomic alterations define major subsets of KRAS-mutant lung adenocarcinoma with distinct biology, immune profiles, and therapeutic vulnerabilities. Cancer Discov 5, 860-77 (2015).

Skoulidis F et al. STK11/LKB1 Mutations and PD-1 Inhibitor Resistance in KRAS-Mutant Lung Adenocarcinoma. Cancer Discov 8, 822-835 (2018).

Kadara H et al. Whole-exome sequencing and immune profiling of early-stage lung adenocarcinoma with fully annotated clinical follow-up. Ann Oncol 28, 75-82 (2017). [PubMed: 27687306].

Scheel A H et al. PD-L1 expression in non-small cell lung cancer: Correlations with genetic alterations. Oncoimmunology 5, e1131379 (2016). [PubMed: 27467949].

Cristescu R et al. Pan-tumor genomic biomarkers for PD-1 checkpoint blockade-based immunotherapy. Science 362 (2018).

Papillon-Cavanagh S, et al. STK11 and KEAP1 mutations as prognostic biomarkers in an observational real-world lung adenocarcinoma cohort. ESMO Open 2020; 5:e000706.

Shire N J et al, STK11 (LKB1) mutations in metastatic NSCLC: Prognostic value in the real world. PLoS ONE (2020) 15 (9): e0238358.

Skoulidis F, Association of STK11/LKB1 genomic alterations with lack of benefit from the addition of pembrolizumab to platinum doublet chemotherapy in non-squamous non-small cell lung cancer. J Clin Oncol (2019).

Hellmann M D et al. Genomic Features of Response to Combination Immunotherapy in Patients with Advanced Non-Small-Cell Lung Cancer. Cancer Cell 33, 843-852 e4 (2018).

Cho B C, et al. Relationship between STK11 and KEAP1 mutational status and efficacy in KEYNOTE-042: pembrolizumab monotherapy versus platinum-based chemotherapy as first-line therapy for PD-L1-positive advanced NSCLC. Cancer Res 2020; 80 (16 Suppl):Abstract nr CT084.

Gadgeel S M, et al. Pembrolizumab Plus Pemetrexed and Platinum versus Placebo Plus Pemetrexed and Platinum as First-Line Therapy For Metastatic Nonsquamous NSCLC: Analysis of KEYNOTE-189 by STK11 and KEAP1 Status. Cancer Res 2020; 80 (16 Suppl):Abstract nr LB-397.

Ricciuti B, Recondo G, Umeton R, et al. Impact of KRAS allele subtypes and concurrent genomic alterations on clinical outcomes to programmed death 1 axis blockade in non-small cell lung cancer. J Clinl Oncol 37 (15)suppl (May 20, 2019).

West H, Cappuzzo F, Reck M, et al. IMpower150: A post hoc analysis of efficacy outcomes in patients with KRAS, STK11 and KEAP1 mutationsAnn Oncol. (2020) 31; Suppl 4, S87-818.

Tate J G, et al. COSMIC: the Catalogue Of Somatic Mutations In Cancer. Nucleic Acids Res (2019) 47 (D1), D941-D947.

Reva B, Antipin Y, Sander C. Predicting the functional impact of protein mutations: application to cancer genomics. Nucleic acids research. 2011; 39 (17):e118.

Sherry, S. T., Ward, M. H., Kholodov, et al. (2001). dbSNP: the NCBI database of genetic variation. Nucleic Acids Res, 29: 308-311

Ausubel et al, Current Protocols in Molecular Biology, John Wiley &amp; Sons, New York, NY, 2003.

Chee et al., Science, 274:610-614, 1996.

Church and Gilbert, Proc. Natl. Acad. Sci. USA 81: 1991-1995, 1988.

Cotton et al., Proc. Natl. Acad. Sci. USA 85:4397-4401, 1985.

Del Tito et al., Clinical Chemistry 44:731-739, 1998.

Drmanac et al., Nat. Biotechnol., 16:54-58, 1998.

Drmanac et al., Science, 260: 1649-1652, 1993.

Eisenhauer E A, et al. New response evaluation criteria in solid tumours: Revised RECIST guideline (version 1.1). Eur J Cancer 45 (2009) 228-247.

Flavell et al, Cell 15:25, 1978.

Geever et al, Proc. Natl. Acad. Sci. USA 78:5081, 1981.

Hellmann et al. Clin Cancer Res.; Jan 15; 24 (2):334-340, 2018.

Johnson et al, Biochem Res Int. 2012:940405, 2012.

Min et al, Molecular Cancer Therapeutics 16:566-577, 2017.

Myers et al, Science 230: 1242, 1985.

Raca et al, Genet Test 8 (4):387-94, 2004.

Remington's Pharmaceutical Sciences 15th Edition.

Sanger et al., Proc. Natl. Acad. Sci. USA 74:5463-5467, 1977.

Schafer et al, Nat. Biotechnol. 15:33-39, 1995.

Sears et al., Biotechniques, 13:626-633, 1992.

Sheffield et al, Proc. Natl. Acad. Sci. USA 86:232-236, 1989.

Underhill et al., Genome Res. 7:996-1005, 1997.

Zaba et al. Lung Cancer., Nov; 82 (2):368-9, 2013.

Zimmerman et al., Methods Mol. Cell. Biol., 3:39-42, 1992.

What is claimed is:

1. A method of treating lung cancer in a subject in need thereof, comprising administering to the subject a therapeutically effective amount of 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1-(2-fluoroprop-2-enoyl)piperazin-2-yl]acetonitrile, or a pharmaceutically acceptable salt thereof, wherein the lung cancer has been determined to have a loss-of-function mutation in STK11 (LKB1), or loss of expression of serine/threonine-protein kinase STK11 (LKB1) protein, and wherein the lung cancer is a KRAS G12C mutant cancer.

2. The method of claim 1, wherein the loss-of-function mutation is a nonsense mutation.

3. The method of claim 1, wherein the loss-of-function mutation is a frameshift mutation.

4. The method of claim 1, wherein the loss-of-function mutation is a splice-site mutation.

5. The method of claim 1, wherein the loss-of-function mutation is a recurrent somatic mutation.

6. The method of claim 1, wherein the lung cancer is a non-small cell lung cancer (NSCLC).

7. The method of claim 1, wherein the subject is a human.

8. The method of claim 7, wherein the human is an adult patient.

9. The method of claim 7, wherein the human is a pediatric patient.

10. The method of claim 1, wherein the therapeutically effective amount of 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1-(2-fluoroprop-2-enoyl)piperazin-2-yl]acetonitrile, or a pharmaceutically acceptable salt thereof, is between about 200 and 1200 mg twice per day (BID).

11. The method of claim 1, wherein the therapeutically effective amount of 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1-(2-fluoroprop-2-enoyl)piperazin-2-yl]acetonitrile, or a pharmaceutically acceptable salt thereof, is about 600 mg twice per day (BID).

12. The method of claim 1, further comprising administering to the subject a second anti-cancer therapy.

13. The method of claim 12, wherein the second anti-cancer therapy is selected from the group consisting of a surgery, an immunotherapy, a radiotherapy, a gene therapy, and a chemotherapy.

14. The method of claim 12, wherein the second anti-cancer therapy is a checkpoint inhibitor.

15. The method of claim 12, wherein the second anti-cancer therapy is a PD-1/PD-L1 inhibitor.

16. The method of claim 15, wherein the PD-1/PD-L1 inhibitor is a PD-1 inhibitor.

17. The method of claim 16, wherein the PD-1 inhibitor is nivolumab, pembrolizumab, cemiplimab or tislelizumab, or a biosimilar thereof.

18. The method of claim 15, wherein the PD-1/PD-L1 inhibitor is a PD-L1 inhibitor.

19. The method of claim 18, wherein the PD-L1 inhibitor is atezolizumab, avelumab, or durvalumab, or a biosimilar thereof.

20. The method of claim 15, wherein the PD-1/PD-L1 inhibitor and the KRAS G12C inhibitor are administered on the same day.

21. The method of claim 15, wherein the PD-1/PD-L1 inhibitor and the KRAS G12C inhibitor are administered on different days.
